(19) United States
(12) Patent Application Publication
(10) Pub. No.: US 2024/0081763 A1
(43) Pub. Date: Mar. 14, 2024

(54) MEDICAL DEVICE OPERATION METHOD AND DRUG TREATMENT SUPPORT SYSTEM

(71) Applicant: Canon Medical Systems Corporation, Otawara-shi (JP)

(72) Inventors: Hiroki TAGUCHI, Otawara (JP); Shinsuke TSUKAGOSHI, Nasushiobara (JP); Yohei MINATOYA, Tokyo (JP)

(73) Assignee: Canon Medical Systems Corporation, Otawara-shi (JP)

(21) Appl. No.: 18/463,384

(22) Filed: Sep. 8, 2023

(30) Foreign Application Priority Data
Sep. 9, 2022 (JP) 2022-143784

(51) Int. Cl. A61B 6/00; A61B 6/03; A61M 5/20

(52) U.S. Cl. CPC A61B 6/5205; A61B 6/032; A61B 6/4241; A61B 6/482; A61M 5/2066

(57) ABSTRACT

According to one embodiment, a medical device operation method includes a monitoring step and an application step. In the monitoring step, a monitoring apparatus executes first CT imaging on a treatment target person into which a first drug particle with a suppressed onset of a treatment effect is introduced, and monitors an accumulation of the first drug particles in a tissue, based on spectral information acquired by the first CT imaging. In the application step, an application apparatus applies an action for exerting the treatment effect of the first drug particle to the first drug particle accumulating in the tissue, by using as a trigger an event that the accumulation of the first drug particles in the tissue satisfies an objective condition.

### CROSS-REFERENCE TO RELATED APPLICATION

This application is based upon and claims the benefit of priority from prior Japanese Patent Application No. 2022-143784, filed Sep. 9, 2022, the entire contents of which are incorporated herein by reference.

### FIELD

Embodiments described herein relate generally to a medical device operation method and a drug treatment support system.

### BACKGROUND

In peripheral blood vessels or new feeding vessels of cancer cells that have progressed to some extent, an EPR (enhanced permeability and retention) effect occurs. The EPR effect is such a phenomenon that there occur an enhancement in vascular permeability due to dilation of gaps between vascular endothelial cells, and an enhancement in accumulation of a vascular permeant substance due to lymphatic system underdevelopment. It is known that the vascular endothelial cell gap is about 5 to 50 nm in normal cells, and is about 100 to 200 nm or more at a time when the EPR effect occurs.

A DDS (drug delivery system) utilizing the EPR effect has been devised. In the DDS utilizing the EPR effect, nano-particles are processed or a drug is sealed by using a liposome or the like, thereby controlling the particle size to a several-hundred nm size, and drug particles are delivered to a cancer tissue. Although the DDS utilizing the EPR effect can deliver drug particles to a cancer tissue, a problem is to exert an effect of a drug, which is sealed for a treatment, at an appropriate timing in the cancer tissue.

### BRIEF DESCRIPTION OF THE DRAWINGS

FIG. 1 is a diagram illustrating a configuration example of a drug treatment support system according to Example 1 of a first embodiment.

FIG. 2 is a view illustrating a cross section of a first drug particle.

FIG. 3 is an explanatory view of a particle size of the first drug particle.

FIG. 4 is a flowchart illustrating a control procedure of various medical devices relating to a drug injection support process according to the first embodiment.

FIG. 5 is a diagram schematically illustrating a processing procedure of a monitoring step according to step S402.

FIG. 6 is an explanatory view of delivery of a second drug particle into a cancer tissue.

FIG. 7 is a diagram illustrating a configuration example of a drug treatment support system 7 according to Example 2.

FIG. 8 is a flowchart illustrating a control procedure of various medical devices relating to a drug injection support process according to Example 2.

FIG. 9 is an explanatory view of breaking of first drug particles by application of high energy.

FIG. 10 is a flowchart illustrating a control procedure of various medical devices relating to a drug injection support process according to Example 3.

FIG. 11 is a diagram schematically illustrating a processing procedure of a monitoring step according to step S1005.

FIG. 12 is an explanatory view of breaking of second drug particles by application of high energy.

FIG. 13 is a diagram illustrating a configuration example of a drug treatment support system according to the first embodiment.

FIG. 14 is a flowchart illustrating a control procedure of various medical devices relating to a drug injection support process according to a second embodiment.

FIG. 15 is a sequence chart of a drug injection support process according to the second embodiment.

### DETAILED DESCRIPTION

According to one embodiment, a medical device operation method includes a monitoring step and an application step. In the monitoring step, a monitoring apparatus executes first CT imaging on a treatment target person into which a first drug particle with a suppressed exertonset of a treatment effect is introduced, and monitors an accumulation of the first drug particles in a tissue, based on spectral information acquired by the first CT imaging. In the application step, an application apparatus applies an action for exerting the treatment effect of the first drug particle to the first drug particle accumulating in the tissue, by using as a trigger an event that the accumulation of the first drug particles in the tissue satisfies an objective condition.

Hereinafter, referring to the accompanying drawings, embodiments of a medical device operation method and a drug treatment support system are described in detail.

### First Embodiment

### Example 1

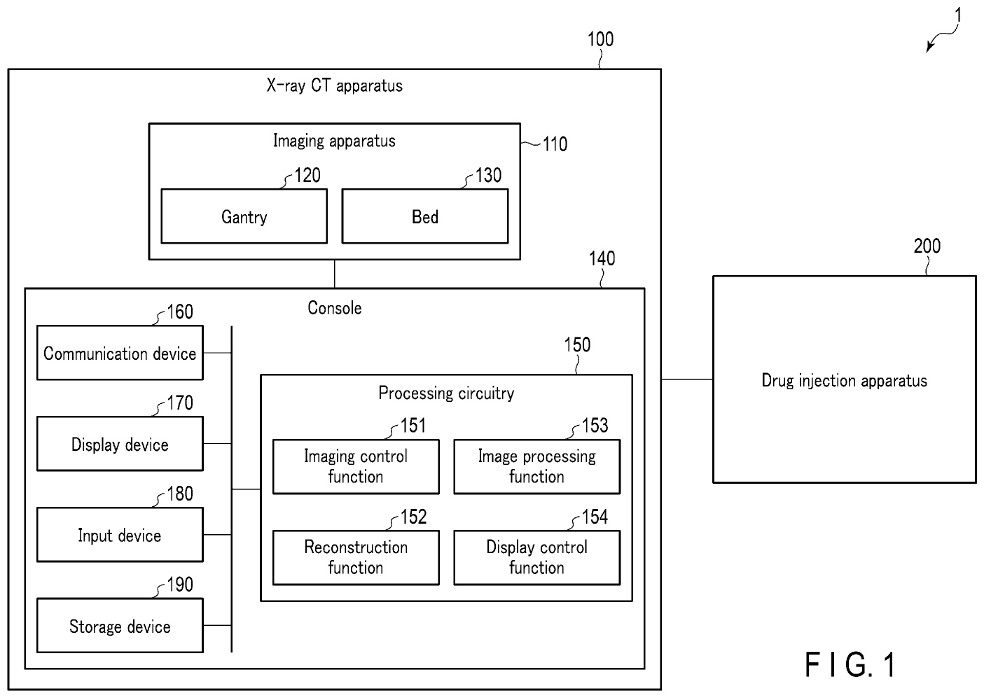

FIG. 1 is a diagram illustrating a configuration example of a drug treatment support system 1 according to Example 1 of a first embodiment. As illustrated in FIG. 1, the drug treatment support system 1 is a network system that supports, by the cooperation of a plurality of medical devices, treating a cancer tissue, which occurs in a treatment target person, by a drug. Specifically, the system includes, as the medical devices, an X-ray CT apparatus (X-ray computed tomography apparatus) 100 and a drug injection apparatus 200. The X-ray CT apparatus 100 and the drug injection apparatus 200 are connected to be capable of mutually communicating information.

As illustrated in FIG. 1, the X-ray CT apparatus 100 includes an imaging apparatus 110 and a console 140, which are connected to be capable of mutually communicating information. The imaging apparatus 110 is a machinery apparatus for executing X-ray CT imaging on a treatment target person. The X-ray CT imaging according to the present embodiment is applicable to any of single energy CT (SECT), dual energy CT (DECT) and photon counting CT (PCCT), and in the description below the photon counting CT (PCCT) is assumed.

The imaging apparatus 110 includes a gantry 120 and a bed 130. The gantry 120 is a substantially cylindrical machinery structure including an opening that forms an imaging space. The gantry 120 includes, in a housing, a rotating frame having a substantially cylindrical shape and a stationary frame that supports the rotating frame such that the rotating frame is rotatable around a rotational axis. Specifically, the stationary frame rotatably supports the rotating frame via a slip ring. In the rotating frame, an X-ray generating device and an X-ray detection device are attached to the rotating frame such that the X-ray generating device and X-ray detection device are opposed to each other, with the rotational axis being interposed.

In the X-ray generating device, an X-ray tube and a high-voltage generator are mounted. The X-ray tube generates X-rays in accordance with the control of tube voltage and tube current by the high-voltage generator. The X-ray detection device includes an X-ray detector and data collection circuitry. The X-ray detector detects X-rays that are generated from the X-ray tube and pass through the treatment target person. In one example, the X-ray detector includes a plurality of semiconductor detection elements formed by compound semiconductors or the like, which are densely arranged in a two-dimensional fashion with respect to a channel direction and a column direction. The semiconductor detection elements capture incident X-ray photons, and convert the X-ray photons into an electric signal having an electric charge corresponding to the energy of the X-ray photons. The data collection circuitry counts X-ray photons, based on the electric signal. In one example, the data collection circuitry counts, by a pulse-height discriminator, the number of X-ray photons in regard to each of a plurality of bins, and collects spectral data representing a distribution of the number (count number) of X-ray photons over the bins. To be more specific, the spectral data is collected in regard to each of views, which mean data sampling points with respect to the X-ray tube rotational direction. Note that the bin means an energy band. The spectral data is transmitted to the console.

The bed 130 includes a table top on which the treatment target person is placed, and a base that movably supports the table top. The treatment target person and the table top are positioned such that a to-be-imaged region of the treatment target person is positioned in the imaging space. Note that the to-be-imaged region according to the present embodiment may be any region if the region includes a tumor, such as a chest part, a waist part, a leg part, an arm part, or a head part. The present embodiment is applicable to both of a benign tumor and a malignant tumor. The malignant tumors can be classified into a carcinoma (malignant epithelial tumor) and a sarcoma (malignant nonepithelial tumor), and the present embodiment is applicable to both malignant tumors. In the description below, it is assumed that the tumor is cancer that is a malignant tumor. A drug for treating a cancer tissue (hereinafter “first drug”) is injected in the treatment target person according to the present embodiment. The first drug is composed of a plurality of nano-particles (hereinafter “first drug particles”) having a treatment effect on the cancer tissue.

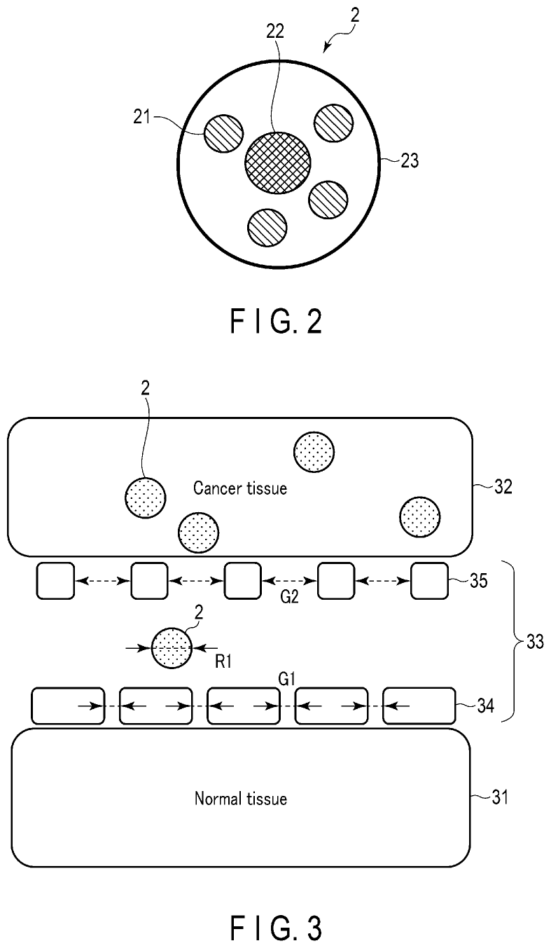

FIG. 2 is a view illustrating a cross section of a first drug particle 2. As illustrated in FIG. 2, the drug particle 2 includes a drug substance 21 and a high atomic number substance 22. The drug substance is a compound having a treatment effect on a cancer tissue. It is assumed that the drug substance 21 includes not only a compound that exerts a treatment effect on the cancer tissue by the substance alone, but also a compound that exerts a treatment effect on the cancer tissue by being affected by some other substance. The high atomic number substance 22 is a metallic substance that is introduced in order to visualize a contrast drug particle by PCCT imaging. The drug substance 21 and high atomic number substance 22 are sealed by a membrane 23. In one example, a lipid membrane, such as a liposome, is used as the membrane 23. It can be said that the treatment effect of the first drug particle 2 is suppressed since the drug substance 21 is sealed by the membrane 23.

The kinds of combinations of the drug substance 21 and high atomic number substance 22 are various in accordance with the kinds of the drug substance 21. For example, it is assumed that the drug substance 21 is a compound that exerts a treatment effect on a cancer tissue by the substance alone, and is, concretely, an anticancer drug. In this case, it is preferable that a substance such as platinum included in the anticancer drug is utilized as the high atomic number substance 22. Note that in a case where the S/N of platinum is low, another heavy metal, such as gold, silver or gadolinium, may be added. In a case where the drug substance 21 is a compound that exerts a treatment effect on a cancer tissue by being affected by some other substance, a freely selected heavy metal, such as gold, silver, gadolinium or platinum, may be utilized.

FIG. 3 is an explanatory view of a particle size of the first drug particle 2. FIG. 3 exemplarily illustrates a normal tissue 31 that is a tissue in which no cancer occurs, and a cancer tissue 32 that is a tissue in which cancer occurs. The normal tissue 31 and cancer tissue 32 are fed by a blood vessel 33. A vessel wall of the blood vessel 33 is composed of vascular endothelial cells 34, 35. Gaps are provided between the vascular endothelial cells 34, 35, and nutritional components or the like flowing in the blood vessel 33 pass through the gaps and are fed to the normal tissue 31 and cancer tissue 32 via interstitial fluid.

With the progress of cancer, the EPR effect occurs in a peripheral blood vessel or new feeding vessel of the cancer tissue 32, the vascular endothelial cells 35 contract, and gaps G2 between the vascular endothelial cells 35 dilate. As illustrated in FIG. 3, a gap G1 between the vascular endothelial cells 34 occupied by the normal tissue 31 is typically about 5 to 50 nm. However, the gap G2 of the vascular endothelial cells 35, in which the EPR effect occurs, is greater than the gap G1, and dilates to about 150 nm or more.

A particle size R1 of the first drug particle 2 is designed such that the first drug particle 2 does not pass through the gap G1 between the vascular endothelial cells 34 in which no EPR effect occurs, but passes through the gap G2 between the vascular endothelial cells 35 in which the EPR effect occurs. According to the above-described example, the particle size R1 of the first drug particle 2 is designed to be about 150 nm or more. By the particle size R1 being designed in this manner, the first drug particle 2 accumulates in the cancer tissue 32.

The console 140 is a computer that controls the imaging apparatus 110. The console 140 includes processing circuitry 150, a communication device 160, a display device 170, an input device 180 and a storage device 190. The processing circuitry 150, communication device 160, display device 170, input device 180 and storage device 190 are connected to be capable of mutually communicating information.

The processing circuitry 150 includes a processor such as a CPU (Central Processing Unit). The processor starts a drug treatment support program that is installed in the storage device 190 or the like, thereby implementing an imaging control function 151, a reconstruction function 152, an image processing function 153 and a display control function 154. The functions 151 to 154 may not necessarily be implemented by single processing circuitry. A plurality of independent processors may be combined to constitute processing circuitry, and the respective processors may implement the functions 151 to 154 by executing the drug treatment support program. In addition, the functions 151 to 154 may be implemented as modules constituting the drug treatment support program, or may be implemented as individual hardware.

By implementing the imaging control function 151, the processing circuitry 150 controls the various devices of the imaging apparatus 110 in accordance with an imaging sequence for PCCT imaging. In accordance with the control by the processing circuitry 150, the imaging apparatus 110 executes PCCT imaging on a to-be-imaged region of the treatment target person, and collects spectral data relating to the to-be-imaged region.

By implementing the reconstruction function 152, the processing circuitry 150 reconstructs image data (hereinafter “PCCT image”) relating to the to-be-imaged region of the treatment target person, based on the collected spectral data. A reconstruction algorithm is not particularly limited, and use can be made of, as appropriate, analytical reconstruction using FBP (filtered back projection), iterative approximation reconstruction that iteratively updates images in such a manner as to optimize an objective function in which various models are formulated, and machine learning reconstruction in which denoise processing by a neural network is assembled in the iterative approximation reconstruction.

By implementing the image processing function 153, the processing circuitry 150 applies various image processes to the PCCT image. In one example, the processing circuitry 150 recognizes, by image processing, an image area corresponding to a drug particle, which is included in the PCCT image.

By implementing the display control function 154, the processing circuitry 150 displays various information on the display device 170. In one example, the processing circuitry 150 displays the PCCT image.

The communication device 160 is an interface for connection to the drug injection apparatus 200, a work station, a PACS (Picture Archiving and Communication System), a HIS (Hospital Information System), a RIS (Radiology Information System), or the like, via a LAN (Local Area Network) or the like. The communication device 160 transmits and receives various data to and from a connection destination.

The display device 170 displays various information in accordance with the display control function 154 of the processing circuitry 150. As the display device 170, use can be made of, as appropriate, a liquid crystal display (LCD), a cathode ray tube (CRT) display, an organic electro-luminescence (EL) display (OELD), a plasma display, or some other freely chosen display. Besides, the display device 170 may be a projector.

The input device 180 accepts various input operations from a user, converts an accepted input operation to an electric signal, and outputs the electric signal to the processing circuitry 150. Specifically, the input device 180 is connected to input devices such as a mouse, a keyboard, a trackball, a switch, a button, a joystick, a touchpad, and a touch panel display. The input device 180 outputs to the processing circuitry 150 an electric signal corresponding to an input operation to the input device 180. In addition, the input device 180 may be an input device provided in some other computer that is connected via a network or the like. The input device 180 may be a speech recognition device that converts a voice signal collected by a microphone into an instruction signal.

The storage device 190 is a storage device that stores various data, such as a ROM (Read Only Memory), a RAM (Random Access Memory), an HDD (Hard Disk Drive), an SSD (Solid State Drive), an integrated circuit storage device, or the like. The storage device 190 stores, for example, a treatment support program or the like. The storage device 190 may be, aside from the above-mentioned storage devices, a portable storage medium such as a CD (Compact Disc), a DVD (Digital Versatile Disc) or a flash memory, or a drive device that reads and writes various information from and to a semiconductor memory element or the like. The storage device 190 may be provided in some other computer that is connected to the X-ray CT apparatus 100 via a network.

The drug injection apparatus 200 is a machinery apparatus that injects a drug particle into a treatment target person. Specifically, the drug injection apparatus 200 includes a cylinder that is a mechanism that discharges a drug particle, and control circuitry that controls the operation of the cylinder by motor driving. Note that the cylinder may be operated based on machine control by the control circuitry, or may be operated manually by a worker.

Next, a drug injection support process by the drug treatment support system 1 is described.

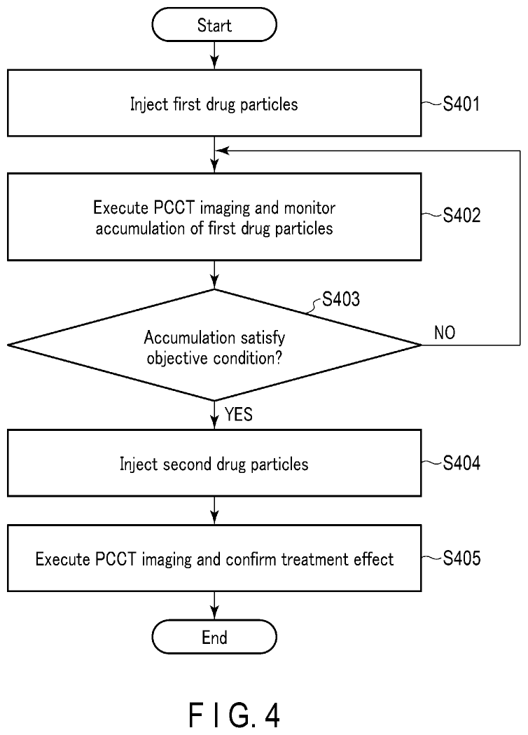

FIG. 4 is a flowchart illustrating a control procedure of various medical devices relating to the drug injection support process according to the first embodiment. Here, as the various medical devices, the X-ray CT apparatus 100 and the drug injection apparatus 200 are assumed.

As illustrated in FIG. 4, the drug injection apparatus 200 injects the first drug particles into the treatment target person (step S401). It is assumed that cancer occurs in the body of the treatment target person. Since the particle size of the first drug particle is designed such that the first drug particle can selectively pass through the gap between vascular endothelial cells in which the EPR effect occurs, the first drug particle is taken in the cancer cell and, accordingly, accumulates in the cancer tissue.

If step S401 is executed, the X-ray CT apparatus 100 executes PCCT imaging, and monitors the accumulation of the first drug particles in the cancer tissue (step S402). If step S402 is executed, the X-ray CT apparatus 100 determines whether the accumulation of the first drug particles in the cancer tissue satisfies an objective condition (step S403). If it is determined that the accumulation of the first drug particles in the cancer tissue fails to satisfy the objective condition (step S403: NO), the X-ray CT apparatus 100 executes PCCT imaging once again, and monitors the accumulation of the first drug particles in the cancer tissue (step S402). In this manner, the X-ray CT apparatus 100 repeats step S402 and step S403 until it is determined in step S403 that the accumulation of the first drug particles in the cancer tissue satisfies the objective condition.

Here, the processing procedure from step S402 to step S403 is described in detail. It is assumed that the drug substance included in the first drug particle is an anticancer drug, and the high atomic number substance is platinum. In addition, it is assumed that an enough amount of first drug particles to satisfy the objective condition in step S403 is injected.

To start with, in step S402, by implementing the imaging control function 151, the processing circuitry 150 controls the imaging apparatus 110 in such a manner as to execute PCCT imaging under a condition for the first drug particle (hereinafter “first condition”). In the first condition, one bin, among a plurality of bins, is set at a local energy band to which a K-edge of the high atomic number substance (platinum) included in the first drug particle belongs. Thereby, by the K-edge imaging utilizing the k-edge of platinum, the first drug particle can be visualized on the PCCT image.

In the PCCT imaging in step S402, a region, in which the occurrence of a cancer tissue is suspected, is set as a to-be-imaged region. The imaging apparatus 110 executes PCCT imaging on the to-be-imaged region at a plurality of time points, and collects spectral data at each time point. Here, it is assumed that the first drug particles cannot immediately accumulate in the cancer tissue. A time (hereinafter “accumulation time”) that is needed for an amount of first drug particles, which is enough to satisfy the objective condition, to accumulate in the cancer tissue varies depending on the kind or the like of the first drug particle, and it is assumed that several minutes, several hours or several days are needed. In a case where the accumulation time is several minutes, the imaging apparatus 110 executes PCCT imaging discretely in a time-series manner or at predetermined time intervals. In this case, the treatment target person is placed on the bed 130. In a case where the accumulation time requires several hours, the imaging apparatus 110 executes PCCT imaging discretely at predetermined time intervals, for example, at intervals of one hour. While the PCCT imaging is not performed, the treatment target person may preferably wait in a waiting room or the like. In a case where the accumulation time requires one day to several days, the imaging apparatus 110 executes PCCT imaging discretely at predetermined time intervals, for example, such as every other day. While the PCCT imaging is not performed, the treatment target person may preferably wait in the home of the treatment target person, a hospital, or the like.

The processing circuitry 150 reconstructs the PCCT image, based on the spectral data at each time point, and displays the reconstructed PCCT image on the display device 170. On the PCCT image, the first drug particle is displayed by being visually distinguished from a cancer tissue, other internal organs, organs, and the like. The processing circuitry 150 monitors the accumulation of the first drug particles in the cancer tissue, based on the PCCT image at each time point. Specifically, the PCCT image is preferably an image by K-edge imaging relating to a bin that is set at the K-edge of the high atomic number substance (platinum).

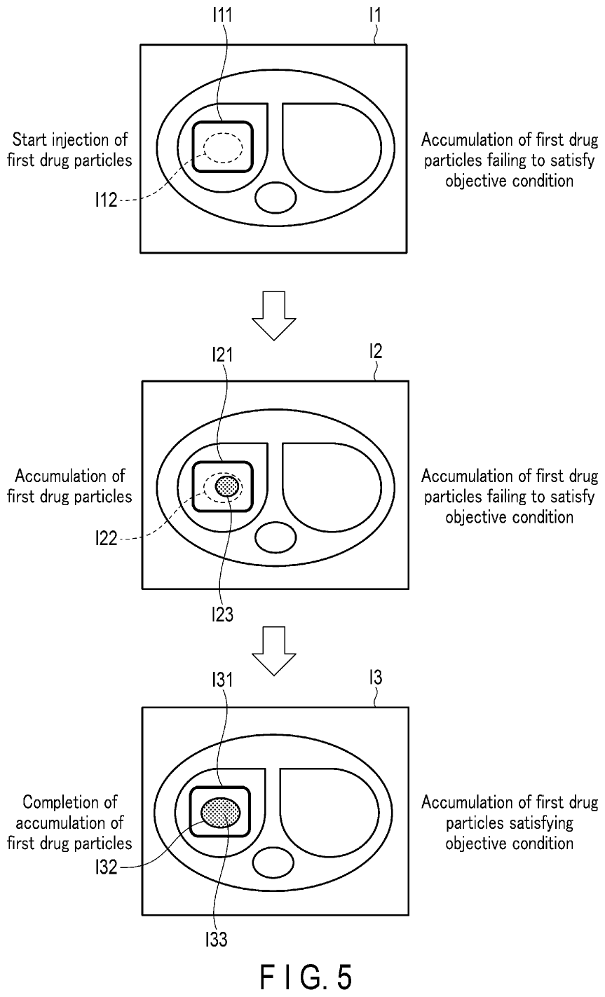

FIG. 5 is a diagram schematically illustrating a processing procedure of a monitoring step according to step S402. A PCCT image I1 illustrated in an upper part of FIG. 5 is a PCCT image at an injection start stage of the first drug particle. A cancer tissue region 112 included in the PCCT image I1 is not opacified by the high atomic number substance of the first drug particle. In order to monitor the accumulation of the first drug particles in the cancer tissue, the processing circuitry 150 computes an index value (hereinafter “accumulation index value”) for quantitatively determining the accumulation of first drug particles in the cancer tissue. For this purpose, the processing circuitry 150 sets a region of interest (ROI) Ill, which is a computation target of the accumulation index value, on the PCCT image I1. The ROI Ill is set in such a manner to surround the cancer tissue region 112. The ROI Ill may be manually set by a worker via the input device 180 or the like, or may be automatically set by image processing on the PCCT image I1.

As the accumulation index value, any index value may be used if the index value can quantitatively determine the accumulation of first drug particles in the cancer tissue. In one example, the number of pixels (pixel number) or the volume of pixels corresponding to the high atomic number substance may preferably be used as the accumulation index value. If it is desired that the first drug particles are distributed at random in the ROI Ill, a standard deviation, a histogram, a texture or the like in the ROI Ill may be used as the accumulation index value.

An objective condition is set for the accumulation index value. It suffices that the objective condition is properly set in accordance with the accumulation index value. In one example, in a case where the accumulation index value is defined by a numerical value of the pixel number, volume, standard deviation or the like, the objective condition may preferably be set at a threshold for the numerical value. In a case where the accumulation index value is defined by a histogram, a texture or the like, the objective condition may preferably be set to be a histogram, texture or the like, which are to be satisfied by the histogram, texture or the like. The objective condition may be empirically determined, or may be individually and specifically set in accordance with a geometrical condition or a clinical condition of the cancer tissue region 112.

If the ROI Ill and the objective condition are set, the processing circuitry 150 computes the accumulation index value, based on the ROI Ill, and determines whether the computed accumulation index value satisfies the objective condition. In the case of the PCCT image I1 of the upper part of FIG. 5, since the first drug particle is not accumulated in the cancer tissue region 112, it is determined that the accumulation index value fails to meet the objective condition. In this case, the PCCT imaging is executed once again after the passage of a predetermined time corresponding to the above-described accumulation time. In this case, the imaging apparatus 110 executes the PCCT imaging, by using as a trigger an event that an imaging start instruction is input by the worker via the input device 180, after the positioning of the treatment target person, table top and the like is finished once again. Note that in a case where it is assumed that the accumulation time is short, the imaging apparatus 110 may execute the PCCT imaging in a time-series manner.

A PCCT image 12 illustrated in a middle part of FIG. 5 is a PCCT image that is generated and displayed while the first drug particles are accumulating. A cancer tissue region 122 included in an ROI 121 is gradually opacified by the first drug particles, and, accordingly, an image area (first drug particle area) 123 corresponding to the first drug particles is extracted. However, the amount of first drug particles that accumulate is small, and the accumulation of the first drug particles is determined to fail to satisfy the objective condition.

A PCCT image 13 illustrated in a lower part of FIG. 5 is a PCCT image that is generated and displayed at an accumulation completion stage of the first drug particles. A most part of a cancer tissue region 132 included in an ROI 131 is extracted by a first drug particle area 133. In this case, the amount of first drug particles that accumulate is large, and the accumulation of the first drug particles is determined to satisfy the objective condition.

If the accumulation of the first drug particles in the cancer tissue is determined to satisfy the objective condition (step S403: YES), the drug injection apparatus 200 injects second drug particles into the treatment target person (step S404). Specific triggers for the injection of the second drug particles by the drug injection apparatus 200 are various. In one example, if the accumulation index value is determined to satisfy the objective condition in step S403, the X-ray CT apparatus 100 automatically transmits an electric signal (hereinafter “injection instruction signal”), which instructs the injection of the second drug particles, to the drug injection apparatus 200. Using the reception of the injection instruction signal as a trigger, the drug injection apparatus 200 injects the second drug particles into the treatment target person. In another example, if the accumulation index value is determined to satisfy the objective condition in step S403, the X-ray CT apparatus 100 causes the display device 170 to display a screen that displays the determination result. The worker that viewed the screen presses an injection start button that is provided on the screen, the imaging apparatus 110 or the like. Using the pressing of the button as a trigger, the X-ray CT apparatus 100 automatically transmits the injection instruction signal to the drug injection apparatus 200. Using the reception of the injection instruction signal as a trigger, the drug injection apparatus 200 injects the second drug particles into the treatment target person. In still another example, the worker, who viewed the screen displaying the determination result indicating that the accumulation index value satisfies the objective condition, may press an injection start button provided on the drug injection apparatus 200. Using the pressing of the button as a trigger, the drug injection apparatus 200 may inject the second drug particles into the treatment target person.

The second drug particle is a drug particle for exerting the treatment effect of the first drug particle. The mechanism for exerting the treatment effect of the first drug particle is not particularly limited. For example, it is assumed that the second drug particle has a property of breaking the membrane of the first drug particle. Like the first drug particle, the second drug particle is labeled with a high atomic number substance. The high atomic number substance of the second drug particle may be of the same kind as, or may be of a different kind from, the high atomic number substance of the first drug particle.

In one example, it is assumed that the membrane of the first drug particle is formed of a liposome having a PH sensitivity. In this case, the second drug particle preferably includes a drug (hereinafter “PH-adjusted drug”) having such a PH as to be capable of breaking the liposome. It is generally known that a cancer tissue or a vicinity thereof has a weakly acidic environment. It is thus preferable that the liposome of the first drug particle is formed of a high polymer that is not broken under a weakly acidic environment and has sensitivity to other PH. The PH-adjusted drug is prepared to have a PH to which the liposome of the first drug particle has sensitivity, such that the PH-adjusted drug can break the liposome of the first drug particle. For example, in a case where the liposome of the first drug particle has sensitivity to weak alkalinity, a weakly alkaline drug is used as the PH-adjusted drug. Preferably, the PH-adjusted drug is sealed by a membrane that is broken in a weakly acidic environment, such that the PH-adjusted drug immediately exerts the effect, when delivered to the cancer tissue.

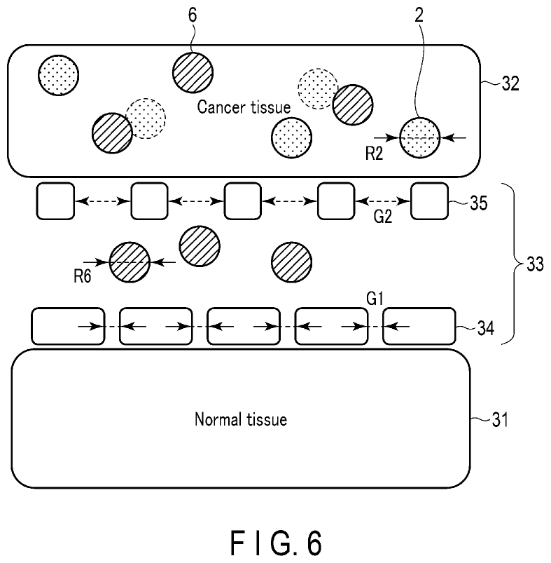

FIG. 6 is an explanatory view of delivery of a second drug particle 6 into the cancer tissue 32. As illustrated in FIG. 6, like the first drug particle 2, the second drug particle 6 is designed to have a particle size R6 such that the second drug particle 6 cannot pass through the gap G1 between the vascular endothelial cells 34 in which no EPR effect occurs, but can pass through the gap G2 between the vascular endothelial cells 35 in which the EPR effect occurs. The particle size R6 of the second drug particle 6 may be, or may not be, identical to a particle size R2 of the first drug particle 2. Thereby, the second drug particles 6 can be delivered to the cancer tissue 32 in which the first drug particles 2 accumulate. If the second drug particles 6 are delivered to the cancer tissue 32, the PH-adjusted drug included in the second drug particle 6 breaks the membrane of the first drug particle 2, and the anticancer drug that is the drug substance is released to the cancer tissue 32. A medical treatment is performed by the released anticancer drug affecting the cancer cells.

By designing the first drug particle and the second drug particle in this manner, it becomes possible to accumulate the first drug particles in the cancer tissue in the state in which the treatment effect of the first drug particles is not exerted, to deliver the second drug particles to the cancer tissue at a timing when the accumulation state becomes appropriate, and to exert the treatment effect of the first drug particle by the chemical action on the first drug particle by the second drug particle. Note that if the influence of the PH-adjusted drug upon the human body is ignorable, there is no need to seal the PH-adjusted drug by the membrane.

If step S404 is executed, the X-ray CT apparatus 100 executes PCCT imaging, and the treatment effect is confirmed (step S405).

Specific triggers for the PCCT imaging in step S405 are various. In one example, if the drug injection apparatus 200 executes the injection of the second drug particles in step S404, the drug injection apparatus 200 automatically transmits an electric signal (hereinafter “imaging instruction signal”), which instructs the execution of PCCT imaging, to the X-ray CT apparatus 100. Using the reception of the imaging instruction signal as a trigger, the X-ray CT apparatus 100 executes PCCT imaging. In another example, if the drug injection apparatus 200 executes the injection of the second drug particles in step S404, the worker presses an imaging start button provided on the X-ray CT apparatus 100. Using the pressing of the button as a trigger, the X-ray CT apparatus 100 executes PCCT imaging.

The PCCT imaging may be executed at a plurality of serial time points or discrete time points, or may be executed only once. By the PCCT imaging, the processing circuitry 150 reconstructs the PCCT image on which the first drug particles and second drug particles are visualized, based on the spectral data, and displays the reconstructed PCCT image on the display device 170. Thereby, the worker can observe the accumulation of the first drug particles and second drug particles in the cancer tissue, and a subsequent treatment effect by the first drug particles.

In a case where the high atomic number substance of the first drug particle and the high atomic number substance of the second drug particle are of the same kind, the spectral data is collected in accordance with the bin that is set at a local energy band to which the K-edge of the high atomic number substance belongs. In this case, the first drug particle and the second drug particle are displayed on the PCCT image without distinction. In a case where the high atomic number substance of the first drug particle and the high atomic number substance of the second drug particle are of different kinds, the spectral data is collected in accordance with a first bin that is set at a local energy band to which the K-edge of the high atomic number substance of the first drug particle belongs, and a second bin that is set at a local energy band to which the K-edge of the high atomic number substance of the second drug particle belongs. In this case, the first drug particle and the second drug particle can distinguishably be displayed on the PCCT image. For example, the processing circuitry 150 displays an image area corresponding to the first drug particle and an image area corresponding to the second drug particle by allocating different color values to these image areas. Thereby, the processing circuitry 150 can display the first drug particles and the second drug particles with different colors on the PCCT image.

By the above, the drug injection support process according to Example 1 is completed.

The above-described drug injection support process according to Example 1 is not limited to the above-described processing procedure, and omission, addition and/or modification can freely be made without departing from the spirit of the invention. In one example, the determination as to whether the accumulation satisfies the objective condition, which is executed in step S403, may be performed by the worker or the like observing the PCCT image or the like. In this case, if a button or the like, which indicates that the objective condition is satisfied, is pressed by the worker via the input device 180, the processing circuitry 150 may determine that the objective condition is satisfied, and if this button is not pressed or if a button or the like, which indicates that the objective condition is not satisfied, is pressed, the processing circuitry 150 may determine that the objective condition is not satisfied.

In another example of the drug injection support process according to Example 1, the structure of the first drug particle is not limited to the above-described structure. Since the high atomic number substance 22 is provided in order to visualize the drug substance or the first drug particle by PCCT imaging, the high atomic number substance 22, for example, may be attached to the membrane that seals the drug substance.

In still another example of the drug injection support process according to Example 1, the drug substance of the first drug particle is not limited to a drug substance that exerts a treatment effect by itself, but may be a drug substance that exerts a treatment effect by reacting with a drug substance of the second drug particle. In this case, the second drug particle includes a drug substance, which is a drug with a suppressed treatment effect on a cancer tissue and exerts a treatment effect on the cancer tissue by reacting with the drug substance of the first drug particle, and a second membrane that seals this drug substance. By the second drug particle being delivered to the cancer tissue, the second membrane is broken, and the drug substance of the first drug particle and the drug substance of the second drug particle react to exert the treatment effect on the cancer tissue. In this case, too, the timing of the exert onset of the treatment effect can freely be controlled.

### Example 2

A drug treatment support system according to Example 2 applies high energy in order to exert the treatment effect of the first drug particle. Hereinafter, the drug treatment support system according to Example 2 is described. In the description below, structural elements having substantially the same functions as in the first embodiment are denoted by like reference signs, and an overlapping description is given only where necessary.

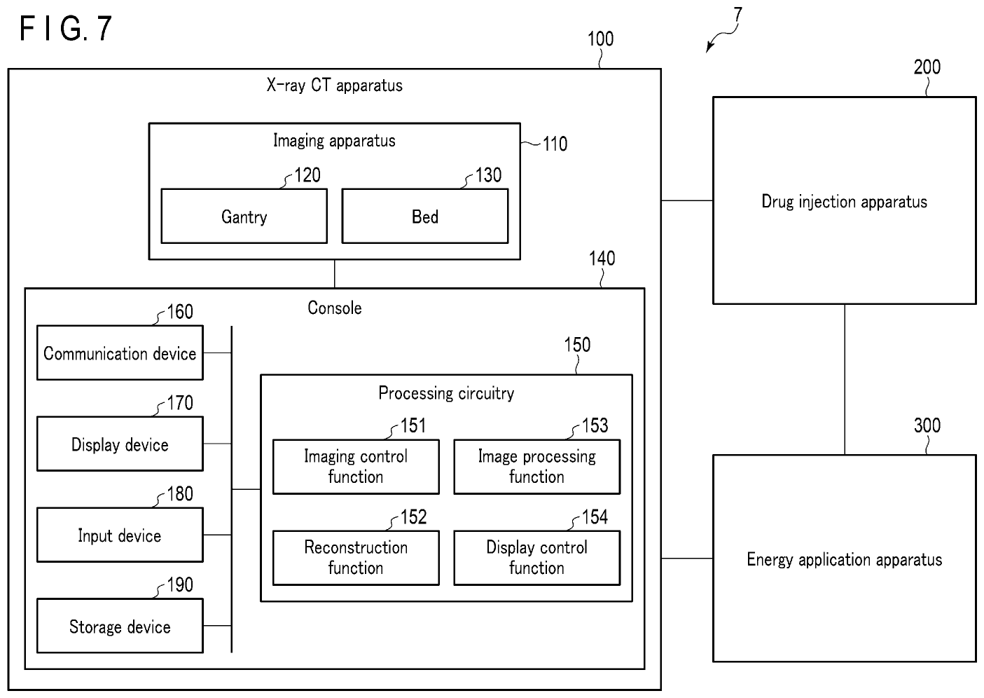

FIG. 7 is a diagram illustrating a configuration example of a drug treatment support system 7 according to Example 2. As illustrated in FIG. 7, specifically, the drug treatment support system 7 includes, as medical devices, an X-ray CT apparatus 100, a drug injection apparatus 200 and an energy application apparatus 300. The X-ray CT apparatus 100, drug injection apparatus 200 and energy application apparatus 300 are connected to be capable of mutually communicating information.

The energy application apparatus 300 is a machinery apparatus that applies high energy. The high energy may be in any form, such as radiation, a magnetic field, or heat, if the high energy can break the membrane that seals the drug substance included in the first drug particle. For example, in a case where the high energy is radiation, an irradiation apparatus is used as the energy application apparatus 300. In a case where the high energy is a magnetic field, a magnetic field generating apparatus such as an electromagnet is used as the energy application apparatus 300. In a case where the high energy is heat, a heat generating apparatus such as a heater is used as the energy application apparatus 300.

Next, a drug injection support process by the drug treatment support system 7 is described.

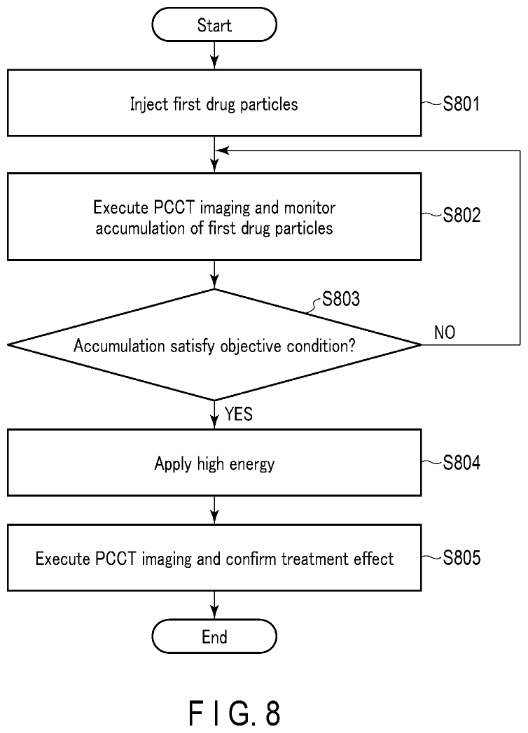

FIG. 8 is a flowchart illustrating a control procedure of various medical devices relating to a drug injection support process according to Example 2. Since steps S801 to S803 are identical to steps S401 to S403 illustrated in FIG. 4, a description thereof is omitted. Note that the structure of the first drug particle according to Example 2 is substantially the same as the structure of the first drug particle illustrated in FIG. 2. However, a different point is that the membrane that seals the drug substance has such a property as to be directly or indirectly breakable by high energy applied by the energy application apparatus 300.

If it is determined in step S803 that the accumulation of the first drug particles in the cancer tissue satisfies the objective condition (step S803: YES), the energy application apparatus 300 applies high energy to the first drug particles accumulating in the cancer tissue (step S804).

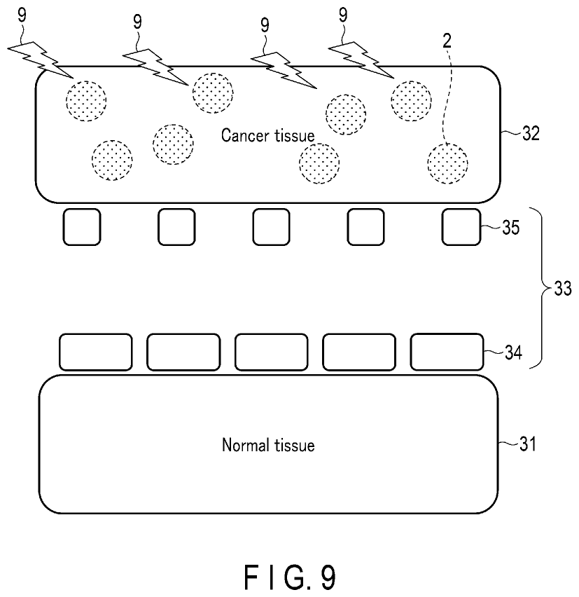

FIG. 9 is an explanatory view of breaking of the first drug particles 2 by application of high energy 9. As illustrated in FIG. 9, the first drug particles 2 accumulate in the cancer tissue 32. In step S804, the energy application apparatus 300 applies the high energy 9 to the first drug particles 2 accumulating in the cancer tissue 32. In one example, in a case where radiation is used as the high energy 9, the energy application apparatus 300 (irradiation apparatus) irradiates a cancer tissue in the body of the treatment target person, or the high atomic number substance of the first drug particle accumulating in the cancer tissue, with high-energy radiation such as X-rays, an electron beam, or a particle beam. Upon receiving the application of the high-energy radiation, the high atomic number substance generates heat or Auger electrons. By the heat or Auger electrons, the membrane of the first drug particle 2 is broken, and the anticancer drug sealed in the membrane is released to the cancer tissue 32. Thereby, the cancer tissue is treated with the anticancer drug.

According to Example 2, the first drug particle is accumulated in the cancer tissue in the state in which the treatment effect of the first drug particle is not exerted, and, by applying high energy to the first drug particle at a timing when the accumulation state becomes appropriate, the membrane of the first drug particle is broken and the treatment effect of the first drug particle can be exerted. Specifically, in Example 2, the onset timing of the treatment effect of the first drug particle is controlled by the application timing of high energy to the first drug particle. Thereby, it is expected that the onset timing can exactly be controlled.

If step S804 is executed, the X-ray CT apparatus 100 executes PCCT imaging, and the treatment effect by the first drug particle is confirmed (step S805). Preferably, the confirmation of the treatment effect according to step S805 is performed by the PCCT image on which the first drug particles are visualized.

By the above, the drug injection support process according to Example 2 is completed.

The above-described drug injection support process according to Example 2 is not limited to the above-described processing procedure, and omission, addition and/or modification can freely be made without departing from the spirit of the invention. In one example, the determination as to whether the accumulation satisfies the objective condition, which is executed in step S803, may be performed by the worker or the like observing the PCCT image or the like. In this case, if a button or the like, which indicates that the objective condition is satisfied, is pressed by the worker via the input device 180, the processing circuitry 150 may determine that the objective condition is satisfied, and if this button is not pressed or if a button or the like, which indicates that the objective condition is not satisfied, is pressed, the processing circuitry 150 may determine that the objective condition is not satisfied.

As another example of the drug injection support process according to Example 2, the energy application apparatus 300 may be a heat generating apparatus. In this case, preferably, the membrane of the first drug particle is formed to have such a property as to be breakable by the heat applied by the energy application apparatus 300. By the membrane of the first drug particle being broken by the heat, the anticancer drug is released, and the cancer tissue can be treated. In this manner, the onset timing of the treatment effect of the first drug particle can be controlled by the heat.

As still another example of the drug injection support process according to Example 2, the first drug particle may not include the drug substance, and may include only the high atomic number substance. In this case, the energy application apparatus 300 applies high energy to the high atomic number substance, and thereby Auger electrons are emitted from the high atomic number substance. It is known that Auger electrons have a treatment effect on cancer. Accordingly, the cancer tissue can be treated by being exposed to Auger electrons. In this manner, the onset timing of the treatment effect of the first drug particle can be controlled by the high energy. Note that in the case of the present modification, it can be said, in other words, that the high atomic number substance doubles as the drug substance.

### Example 3

A drug treatment support system according to Example 3 uses the injection of the second drug particle and the application of high energy in order to exert the treatment effect of the first drug particle. Hereinafter, the drug treatment support system according to Example 3 is described. In the description below, structural elements having substantially the same functions as in Example 1 and Example 2 are denoted by like reference signs, and an overlapping description is given only where necessary.

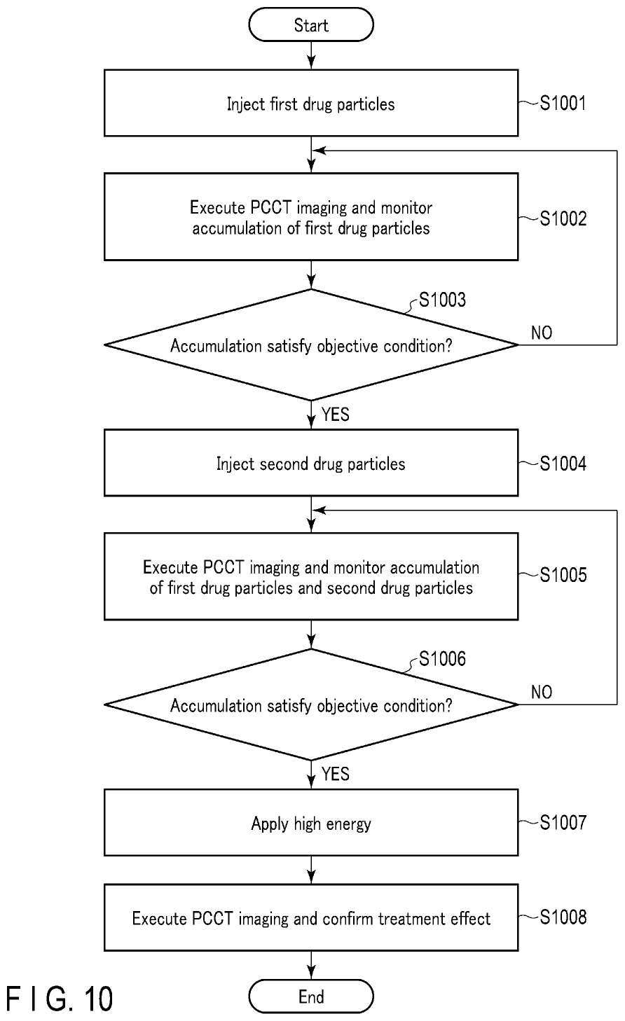

FIG. 10 is a flowchart illustrating a control procedure of various medical devices relating to a drug injection support process according to Example 3. Note that the configuration of the drug treatment support system according to Example 3 is the same as in Example 2. In addition, since steps S1001 to S1003 are identical to steps S401 to S403 illustrated in FIG. 4, a description thereof is omitted.

If the accumulation of the first drug particles in the cancer tissue is determined to satisfy the objective condition in step S1003 (step S1003: YES), the drug injection apparatus 200 injects second drug particles into the treatment target person. The second drug particle according to Example 3 includes a drug substance for breaking the membrane of the first drug particle, and a membrane that seals this drug substance. The membrane has such a property as to be directly or indirectly breakable by the application of high energy by the energy application apparatus 300.

If step S1004 is executed, the X-ray CT apparatus 100 executes PCCT imaging, and monitors the accumulation of the first drug particles and second drug particles (step S1005). If step S1005 is executed, the X-ray CT apparatus 100 determines whether the accumulation of the first drug particles and second drug particles in the cancer tissue satisfies an objective condition (step S1006). If it is determined that the accumulation of the first drug particles and second drug particles in the cancer tissue fails to satisfy the objective condition (step S1006: NO), the X-ray CT apparatus 100 executes PCCT imaging once again, and monitors the accumulation of the first drug particles and second drug particles in the cancer tissue (step S1005). In this manner, the X-ray CT apparatus 100 repeats step S1005 and step S1006 until it is determined in step S1006 that the accumulation of the first drug particles and second drug particles in the cancer tissue satisfies the objective condition.

Here, the processing procedure from step S1005 to step S1006 is described in detail. It is assumed that the drug substance included in the first drug particle is an anticancer drug, and the high atomic number substance is platinum. In addition, it is assumed that the drug substance included in the second drug particle is a PH-adjusted drug that breaks the membrane of the first drug particle, the high atomic number substance is gold, and the membrane has such a property as to be breakable by radiation. To start with, in step S1005, by implementing the imaging control function 151, the processing circuitry 150 controls the imaging apparatus 110 in such a manner as to execute PCCT imaging under a condition for the second drug particle (hereinafter “second condition”). In the second condition, one bin, among a plurality of bins, is set at a local energy band to which a K-edge of the high atomic number substance (gold) included in the second drug particle belongs. Thereby, the second drug particle can selectively be visualized on the PCCT image.

In the PCCT imaging in step S1005, a region, in which the occurrence of a cancer tissue is suspected, is set as a to-be-imaged region. The imaging apparatus 110 executes PCCT imaging on the to-be-imaged region at a plurality of time points, and collects spectral data at each time point. The processing circuitry 150 reconstructs the PCCT image, based on the spectral data at each time point, and displays the reconstructed PCCT image on the display device 170. On the PCCT image, the second drug particle is displayed by being visually distinguished from a cancer tissue, other internal organs, organs, and the like. Specifically, the PCCT image is preferably an image by K-edge imaging relating to a bin that is set at the K-edge of the high atomic number substance (gold). Based on the PCCT image at each time point, the processing circuitry 150 monitors the accumulation of the second drug particles in the cancer tissue.

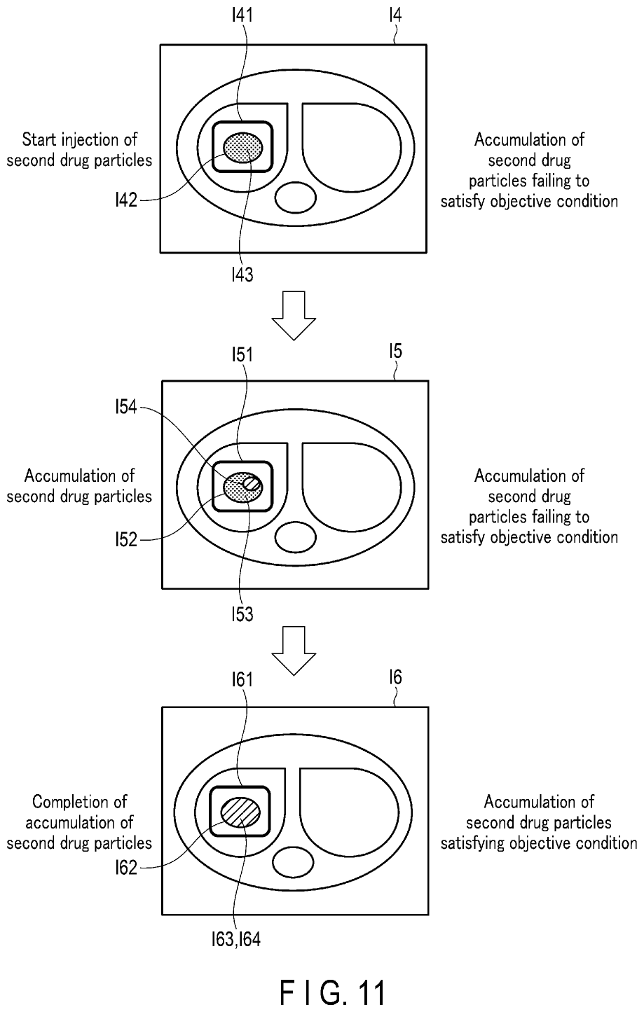

FIG. 11 is a diagram schematically illustrating a processing procedure of a monitoring step according to step S1005. A PCCT image 14 illustrated in an upper part of FIG. 11 is a PCCT image at an injection start stage of the second drug particle. A cancer tissue region 142 included in the PCCT image 14 is opacified by the high atomic number substance of the first drug particle. In order to monitor the accumulation of the second drug particles in the cancer tissue, the processing circuitry 150 computes an accumulation index value for quantitatively determining the accumulation of the second drug particles in the cancer tissue. For this purpose, the processing circuitry 150 sets an ROI 141, which is a computation target of the accumulation index value, on the PCCT image 14. The ROI 141 is set in such a manner to surround the cancer tissue region 142. The ROI 141 may be manually set by the worker via the input device 180 or the like, or may be automatically set by image processing on the PCCT image 14. The accumulation index value and an objective condition relating to the second drug particle may be set similarly to those relating to the first drug particle.

If the ROI 141 and the objective condition are set, the processing circuitry 150 computes the accumulation index value, based on the ROI 141, and determines whether the computed accumulation index value satisfies the objective condition. In the case of the PCCT image 14 of the upper part of FIG. 11, since the second drug particle is not accumulated in the cancer tissue region 142, it is determined that the accumulation index value fails to meet the objective condition. In this case, the PCCT imaging is executed once again after the passage of a freely selected time.

A PCCT image 15 illustrated in a middle part of FIG. 11 is a PCCT image that is generated and displayed while the second drug particles are accumulating. A cancer tissue region 152 included in an ROI 151 is gradually opacified by the second drug particles, and an image area (second drug particle area) 153 corresponding to the second drug particles is extracted. However, since the amount of second drug particles that accumulate is small, the accumulation of the second drug particles is determined to fail to satisfy the objective condition.

A PCCT image 16 illustrated in a lower part of FIG. 11 is a PCCT image that is generated and displayed at an accumulation completion stage of the second drug particles. A most part of a cancer tissue region 162 included in an ROI 161 is extracted by a second drug particle area 163. In this case, the amount of second drug particles that accumulate is large, and the accumulation of the second drug particles is determined to satisfy the objective condition.

If the accumulation of the second drug particles in the cancer tissue is determined to satisfy the objective condition (step S1006: YES), the energy application apparatus 300 applies high energy to the second drug particles accumulating in the cancer tissue (step S1007).

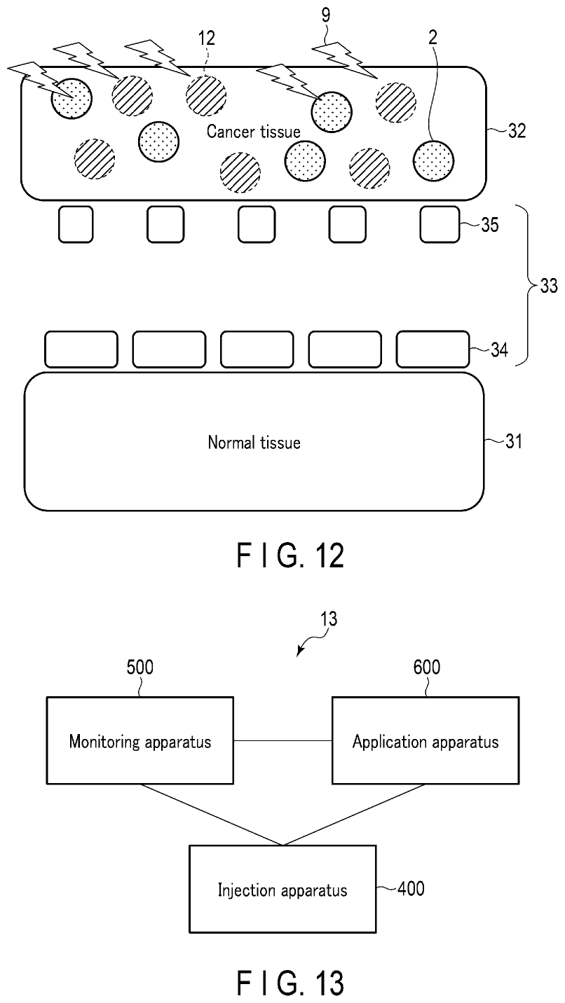

FIG. 12 is an explanatory view of breaking of second drug particles 12 by application of high energy 9. As illustrated in FIG. 12, the first drug particles 2 and second drug particles 12 accumulate in the cancer tissue 32. In step S1007, the energy application apparatus 300 applies the high energy 9 to the second drug particles 2 accumulating in the cancer tissue 32. In one example, in a case where radiation is used as the high energy 9, the energy application apparatus 300 irradiates a cancer tissue in the body of the treatment target person, or the second drug particles 12 accumulating in the cancer tissue, with high-energy radiation such as X-rays, an electron beam, or a particle beam. Thereby, the membrane of the second drug particle 12 is broken, and the PH-adjusted drug sealed in the membrane is released to the cancer tissue 32. By the released PH-adjusted drug, the membrane of the first drug particle 2 is broken, and the anticancer drug sealed in the membrane is released to the cancer tissue 32, and the cancer tissue 32 is treated with the anticancer drug.

According to Example 3, by applying the high energy to the second drug particles at a timing when the accumulation state of the second drug particles becomes appropriate, the treatment effect of the first drug particles can indirectly be exerted. Accordingly, in Example 3, compared to Example 1 or Example 2 in which the onset timing of the treatment effect of the first drug particle is controlled by the injection timing of the second drug particle or the application timing of the energy, it is expected that the onset timing can more exactly be controlled.

If step S1007 is executed, the X-ray CT apparatus 100 executes PCCT imaging, and the treatment effect by the first drug particles is confirmed (step S1008). The confirmation of the treatment effect is performed by the PCCT image on which the first drug particles and second drug particles are visualized.

<Conclusion>

Here, a conclusion is given on the drug treatment support system according to the first embodiment.

FIG. 13 is a diagram illustrating a configuration example of a drug treatment support system 13 according to the first embodiment. As illustrated in FIG. 13, the drug treatment support system 13 according to the first embodiment includes an injection apparatus 400, a monitoring apparatus 500 and an application apparatus 600. The injection apparatus 400 injects a first drug particle with suppressed onset of a treatment effect into a treatment target person. The monitoring apparatus 500 executes a monitoring step of executing first CT imaging on the treatment target person into which the first drug particles with the suppressed onset of the treatment effect is introduced, and monitoring an accumulation of the first drug particles in a tissue, based on spectral information acquired by the first CT imaging. By using as a trigger an event that the accumulation of the first drug particles in the tissue satisfies an objective condition, the application apparatus 600 applies an action for exerting the treatment effect of the first drug particles to the first drug particles accumulating in the tissue.

In the above-described first embodiment, the tissue is assumed to be a cancer tissue. However, the present embodiment is not limited to this. The tissue according to the first embodiment may be any tissue if the tissue is a biological tissue having a property of accumulating a drug or a drug particle.

In the above-described Examples 1 to 3, it is assumed that the X-ray CT apparatus 100 that executes PCCT imaging is used as the monitoring apparatus 500. In this case, the spectral information corresponds to a PCCT image or PCCT scan data collected by PCCT imaging executed by the X-ray CT apparatus 100. The spectral information is not limited to this, and, as the spectral information, use may be made of a SECT image or SECT scan data collected by SECT imaging executed by the X-ray CT apparatus 100. In this case, the processing circuitry 150 can generate a PCCT image by applying a SECT image or SECT scan data collected by the SECT imaging to a neural network. In another example, the spectral information may be a PCCT image based on a DECT image or DECT scan data collected by DECT imaging executed by the X-ray CT apparatus 100. In this case, the processing circuitry 150 can generate a PCCT image by applying the DECT image or DECT scan data collected by the DECT imaging to a neural network.

Since it suffices that the monitoring apparatus 500 can visualize the accumulation of the first drug particles and second drug particles in the tissue, the monitoring apparatus 500 may be an image diagnosis apparatus other than the X-ray CT apparatus, such as a magnetic resonance imaging apparatus, an ultrasonic diagnosis apparatus, a PET (Positron Emission Tomography) apparatus, or a SPECT (Single Photon Emission CT) apparatus.

In Example 1, the application apparatus 600 is the drug injection apparatus 200. In the application step, using as a trigger an event that the objective condition is satisfied, the drug injection apparatus 200 injects the second drug particle into the treatment target person, and the treatment effect of the first drug particle is exerted by the action of the second drug particle upon the first drug particle. In one example, the first drug particle includes a drug substance for treating a tissue, and a membrane that seals the drug substance. In this case, in the application step, the membrane is broken by a physical or chemical action between the second drug particle and the first drug particle, and the drug substance is released. In another example, the first drug particle includes a first drug with a suppressed treatment effect on a tissue, and a first membrane that seals the first drug, and the second drug particle includes a second drug, which is a drug with a suppressed treatment effect on a tissue and exerts a treatment effect on the tissue by reacting with the first drug, and a second membrane that seals the second drug. In this case, in the application step, the first membrane and the second membrane are broken in the tissue, and the first drug and the second drug react to exert the treatment effect on the tissue. The injection apparatus 400 may be identical to, or different from, the drug injection apparatus 200.

According to Example 1, the first drug particles are accumulated in the tissue in the state in which the treatment effect of the first drug particles is not exerted, the second drug particles are delivered to the tissue at a timing when the accumulation state becomes appropriate, and the treatment effect of the first drug particle is exerted by the chemical action on the first drug particle by the second drug particle. Thereby, the treatment effect of the first drug particle can be exerted at an appropriate timing in the tissue.

In Example 2, the application apparatus 600 is the energy application apparatus 300. In the application step, using as a trigger an event that the objective condition is satisfied, the energy application apparatus 300 applies energy to the treatment target person, and the treatment effect of the first drug particles is exerted by the action of the applied energy upon the first drug particles. In one example, the first drug particle includes a drug substance for treating a tissue, a high atomic number substance, and a membrane that seals the drug substance and the high atomic number substance. In this case, in the application step, the energy is applied to the high atomic number substance to generate heat and/or Auger electrons, and the membrane is broken by the heat and/or Auger electrons to release the drug substance. In another example, the first drug particle includes a high atomic number substance. In this case, in the application step, energy is applied to the high atomic number substance, thereby generating Auger electrons that exert a treatment effect on the tissue.

According to Example 2, the first drug particles are accumulated in the tissue in the state in which the treatment effect of the first drug particles is not exerted, and high energy is applied to the first drug particles at a timing when the accumulation state becomes appropriate, thereby exerting the treatment effect of the first drug particles. Thereby, the treatment effect of the first drug particles can be exerted at an appropriate timing in the tissue.

The monitoring apparatus 500 according to the first embodiment further executes a determination step of computing the accumulation index value, based on the spectral information, and determining whether the accumulation index value satisfies the objective condition. In one example, the objective condition is set to be that the accumulation index value of the first drug particles has reached a predetermined value. In the application step, if it is determined that the accumulation index value satisfies the objective condition, an action is applied to the first drug particles. The monitoring step is not necessarily executed by the monitoring apparatus 500, and may be executed by an image processing apparatus or the like, which is different from the monitoring apparatus 500.

The monitoring apparatus 500 according to the first embodiment may further execute a confirmation step of executing medical imaging on the treatment target person during and/or after the application of the action, and confirming the treatment effect by the first drug particles, based on medical imaging information acquired by the medical imaging. In Examples 1 to 3, the monitoring apparatus 500 is the X-ray CT apparatus 100, and the medical imaging information is the spectral information. The confirmation step may be executed by a monitoring apparatus that is different from the monitoring apparatus 500 that executes the monitoring step. As the monitoring apparatus, use can be made of a medical image diagnosis apparatus such as a magnetic resonance imaging apparatus, an ultrasonic diagnosis apparatus, a PET apparatus, or a SPECT apparatus. In this case, the medical imaging information corresponds to an image collected by various medical image diagnosis apparatuses, or raw data used for reconstruction of the image.

According to the first embodiment, the first drug particles are accumulated in the tissue in the state in which the treatment effect of the first drug particles is not exerted, and an action is applied to the first drug particles at a timing when the accumulation state becomes appropriate, thus being able to exert the treatment effect of the first drug particles. Thereby, the treatment effect of the first drug particles can be exerted at an appropriate timing in the tissue, and, by extension, it is expected that the treatment effect of the drug treatment is improved.

### Second Embodiment

A drug treatment support system according to a second embodiment repeats the injection of first drug particles and the monitoring of the accumulation of the first drug particles until the accumulation of the first drug particles in a tissue satisfies an objective condition. Hereinafter, the drug treatment support system according to the second embodiment is described. In the description below, structural elements having substantially the same functions as in the first embodiment are denoted by like reference signs, and an overlapping description is given only where necessary. In addition, like the first embodiment, the tissue according to the second embodiment may be any tissue if the tissue is a biological tissue having a property of accumulating a drug or a drug particle. In one example, in the following description of the second embodiment, it is assumed that the tissue is a cancer tissue.

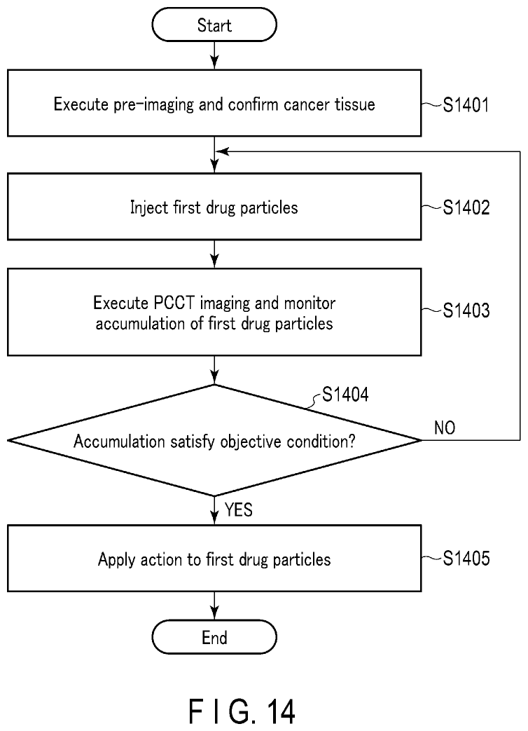

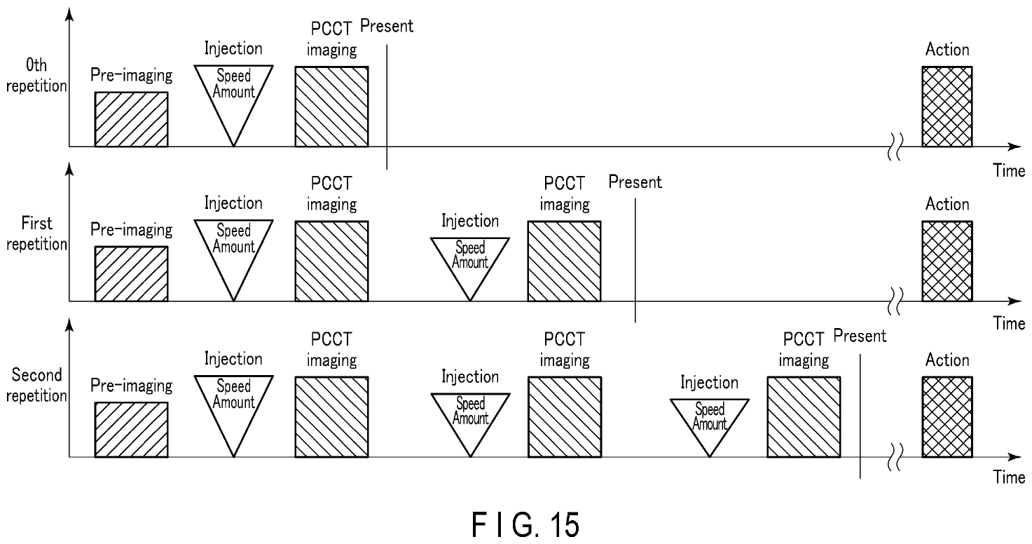

FIG. 14 is a flowchart illustrating a control procedure of various medical devices relating to a drug injection support process according to the second embodiment. Note that it is assumed that the configuration of the drug treatment support system according to the second embodiment is the same as the configuration of the drug treatment support system 13 illustrated in FIG. 13. It is assumed that the monitoring apparatus 500 is the X-ray CT apparatus 100. FIG. 15 is a sequence chart relating to the drug injection support process illustrated in FIG. 14. FIG. 15 exemplarily illustrates execution timings of pre-imaging (step S1401), injection of first drug particles (step S1402), PCCT imaging (step S1403) and application of an action (step S1405) at times when the number of times of repetition of steps S1402 to S1404 illustrated in FIG. 14 is 0, 1 and 2.

As illustrated in FIG. 14, to start with, the monitoring apparatus 500 executes pre-imaging and confirms a cancer tissue (step S1401). Specifically, in step S1401, the monitoring apparatus 500 executes PCCT imaging on the treatment target person, collects spectral data, reconstructs the PCCT image, based on the collected spectral data, and displays the reconstructed PCCT image. The worker observes the PCCT image, and confirms tumor characteristics of the cancer tissue of the treatment target person, such a progress degree, type, position and size. Then, the worker determines an injection speed and an injection amount of first drug particles to be injected in step S1402, in accordance with the tumor characteristics. The injection speed and injection amount may be determined by applying, by the processing circuitry 150, the tumor characteristics of the cancer tissue of the treatment target person to a machine learning model, or may be determined by applying, by the processing circuitry 150, the PCCT image to a machine learning model. Here, as the machine learning model, a neural network, a support vector machine, clustering, LUT (look up table) can be used. The injection speed and injection amount may also be determined by taking into account a load on the treatment target person, and the like, in addition to the tumor characteristics and the PCCT image.

If step S1401 is executed, the injection apparatus 400 injects the first drug particles into the treatment target person (step S1402). The injection apparatus 400 injects the first drug particles in accordance with the injection speed and injection amount determined in step S1401.

If step S1402 is executed, the monitoring apparatus 500 executes PCCT imaging, and monitors the accumulation of the first drug particles in the cancer tissue (step S1403). Specifically, in step S1403, the monitoring apparatus 500 executes PCCT imaging on the treatment target person, collects spectral data, reconstructs the PCCT image, based on the collected spectral data, and displays the reconstructed PCCT image. In one example, as the PCCT imaging, use may be made of K-edge imaging utilizing the k-edge of the high atomic number substance included in the first drug particle. The processing circuitry 150 of the monitoring apparatus 500 image-processes the PCCT image and recognizes the first drug particles by implementing the image processing function 153, and displays the first drug particles on the display device 170 with emphasis by a color or the like, by implementing the display control function 154. Thereby, the accumulation of the first drug particles in the cancer tissue is visualized on the PCCT image. By observing the PCCT image, the worker can visually confirm the accumulation of the first drug particles in the cancer tissue. Note that it is also possible to confirm that the first drug particles are not accumulated in the normal tissue.

In addition, in order to monitor the accumulation of the first drug particles in the cancer tissue, the processing circuitry 150 sets the ROI in such a manner to surround the cancer tissue region in the PCCT image, and computes the accumulation index value for quantitatively determining the accumulation of first drug particles in the cancer tissue region included in the ROI. The computation method of the accumulation index value is similar to that in the first embodiment, and any one is freely set from among the pixel number, volume, standard deviation, histogram, texture, and the like, of the first drug particles.

If step S1403 is executed, the monitoring apparatus 500 determines whether the accumulation of the first drug particles in the cancer tissue satisfies the objective condition (step S1404). Specifically, in step S1404, the processing circuitry 150 of the monitoring apparatus 500 determines whether the accumulation index value satisfies the objective condition. The objective condition is set to be an accumulation index value at a time when the mode of the accumulation of the first drug particles in the cancer tissue is ideal.

If it is determined in step S1404 that the accumulation of the first drug particles in the cancer tissue fails to satisfy the objective condition (step S1404: NO), the injection apparatus 400 injects the first drug particles into the treatment target person once again (step S1402), and the monitoring apparatus 500 executes PCCT imaging and monitors the accumulation of the first drug particles (step S1403), and determines whether the accumulation satisfies the objective condition (step S1404). In this manner, steps S1402 to S1404 are repeated until it is determined in step S1404 that the accumulation of the first drug particles in the cancer tissue satisfies the objective condition.

Typically, the injection speed and injection amount relating to an n-th injection (n: a natural number of 2 or more) of the first drug particles are set to be different values from the injection speed and injection amount of an (n−1)th injection of the first drug particles. Like the above-described step S1403, the injection speed and injection amount of the n-th injection can be determined based on the PCCT image acquired by the (n−1)th PCCT imaging. A time interval between the n-th PCCT imaging and the (n−1)th PCCT imaging may freely be set in consideration of the accumulation characteristic of the first drug particles, the load on the treatment target person, and the like. In one example, it is assumed that the injection of the first drug particles is performed in units of several hours to several days.

If it is determined in step S1404 that the accumulation of the first drug particles in the cancer tissue satisfies the objective condition (step S1404: YES), the application apparatus 600 applies an action to the first drug particles (step S1405). The action may be any of the kinds described in the first embodiment. By the action being applied, the treatment effect of the first drug particles, which are appropriately accumulated in the cancer tissue, is exerted.

By the above, the drug injection support process according the second embodiment is completed.

Note that the above-described drug injection support process according to the second embodiment is not limited to the above-described processing procedure, and omission, addition and/or modification can freely be made without departing from the spirit of the invention. In one example, the determination as to whether the accumulation satisfies the objective condition, which is executed in step S1404, may be performed by the worker or the like observing the PCCT image or the like. In this case, if a button or the like, which indicates that the objective condition is satisfied, is pressed by the worker via the input device 180, the processing circuitry 150 may determine that the objective condition is satisfied, and if this button is not pressed or if a button or the like, which indicates that the objective condition is not satisfied, is pressed, the processing circuitry 150 may determine that the objective condition is not satisfied.

In another example, in the above-described drug injection support process, although the possibility/impossibility of the application of the action was described as being determined based on the accumulation of the first drug particles in the cancer tissue, the possibility/impossibility of the application of the action may be determined by also taking into account the accumulation of the first drug particles in the normal tissue. In one example, if the accumulation of the first drug particles in the cancer tissue satisfies the objective condition and if the accumulation of the first drug particles in the normal tissue is less than a threshold condition, it may be determined that the application of the action is possible. The threshold condition may be zero, or a freely set value greater than zero.

As described above, the drug treatment support system according to the second embodiment includes the injection apparatus 400, monitoring apparatus 500 and application apparatus 600. The injection apparatus 400 executes the injection step (S1402) of injecting the first drug particles into the treatment target person. The monitoring apparatus 500 executes the monitoring step (S1403) of executing medical imaging on the treatment target person and monitoring the accumulation of the first drug particles in the tissue, based on the spectral information acquired by the CT imaging. Further, the monitoring apparatus 500 executes the repetition step (S1402 to S1404) of repeating the injection step and the monitoring step until the accumulation of the first drug particles in the tissue satisfies the objective condition.

In one example, in the repetition step, the accumulation index value is computed by image-processing the spectral information, and the objective condition is determined to be satisfied in a case where the accumulation index value reaches a predetermined value. In another example, in the repetition step, the objective condition is determined to be satisfied in a case where the worker inputs an instruction indicating the satisfaction of the objective condition via the input device 180.

According to the second embodiment, the injection step of the first drug particles and the monitoring step are repeated such that the first drug particles are appropriately accumulated in the tissue, and the treatment effect of the first drug particles can be exerted at a timing when the first drug particles are appropriately accumulated in the tissue. In addition, since the injection speed and injection mount of the first drug particles can be appropriately controlled, it is expected that side effects by the first drug particles are reduced.

According to at least one of the above-described embodiments, the treatment effect of the drug can be exerted at an appropriate timing in the tissue having a property of accumulating the drug.

The term “processor” used in the above description means, for example, circuitry such as a CPU, a GPU, an application specific integrated circuit (ASIC), or a programmable logic device (for example, a simple programmable logic device (SPLD), a complex programmable logic device (CPLD) or a field programmable gate array (FPGA)). The processor implements functions by reading and executing a program stored in the storage circuitry. Note that, instead of storing the program in the storage circuitry, such a configuration may be adopted that the program is directly assembled in the circuitry of the processor. In this case, the processor implements functions by reading and executing the program assembled in the circuitry of the processor. On the other hand, in a case where the processor is, for example, an ASIC, the functions are directly assembled as logic circuitry in the circuitry of the processor, instead of the program being stored in the storage circuitry. Note that the processors in the embodiments are not limited to cases where each processor is constituted as single circuitry, and a plurality of independent circuities may be combined to constitute one processor and to implement functions thereof. Furthermore, a plurality of constituent elements in FIG. 1, FIG. 7 and FIG. 13 may be integrated into one processor, and the processor may implement the functions thereof.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical device operation method comprising:
a monitoring step of executing, by an X-ray CT apparatus, first CT imaging on a treatment target person into which a first drug particle with a suppressed onset of a treatment effect is introduced, and monitoring an accumulation of the first drug particles in a tissue, based on spectral information acquired by the first CT imaging; and
an application step of applying, by an application apparatus, an action for exerting the treatment effect of the first drug particle to the first drug particle accumulating in the tissue, by using as a trigger an event that the accumulation of the first drug particles in the tissue satisfies an objective condition.

2. The medical device operation method of claim 1, wherein
the application apparatus is a drug injection apparatus, and
the application step includes injecting, by the drug injection apparatus, a second drug particle into the treatment target person, by using as a trigger an event that the objective condition is satisfied, and exerting the treatment effect of the first drug particle by the action of the second drug particle upon the first drug particle.

3. The medical device operation method of claim 2, wherein
the first drug particle includes a drug for treating the tissue and a membrane that seals the drug, and
the application step includes breaking the membrane by the action, which is physical or chemical, between the second drug particle and the first drug particle, and releasing the drug.

4. The medical device operation method of claim 2, wherein
the first drug particle includes a first drug with a suppressed treatment effect on the tissue, and a first membrane that seals the first drug,
the second drug particle includes a second drug, which is a drug with a suppressed treatment effect on the tissue and exerts a treatment effect on the tissue by reacting with the first drug, and a second membrane that seals the second drug, and
the application step includes breaking the second membrane and the first membrane in the tissue, and exerting the treatment effect on the tissue by a reaction between the first drug and the second drug.

5. The medical device operation method of claim 1, wherein
the application apparatus is an energy application apparatus, and
the application step includes applying, by the energy application apparatus, energy to the treatment target person, by using as a trigger an event that the objective condition is satisfied, and exerting the treatment effect of the first drug particle by the action of the energy upon the first drug particle.

6. The medical device operation method of claim 5, wherein
the first drug particle includes a drug for treating the tissue, a high atomic number substance, and a membrane that seals the drug and the high atomic number substance, and
the application step includes generating heat and/or Auger electrons by applying the energy to the high atomic number substance, and releasing the drug by breaking the membrane by the heat and/or Auger electrons.

7. The medical device operation method of claim 5, wherein
the first drug particle includes a high atomic number substance, and
the application step includes applying the energy to the high atomic number substance, thereby generating Auger electrons that exert the treatment effect on the tissue.

8. The medical device operation method of claim 1, wherein
the first drug particle is labeled with a high atomic number substance, and
the first CT imaging is K-edge imaging utilizing a K-edge of the high atomic number substance.

9. The medical device operation method of claim 8, further comprising a determination step, wherein
the determination step includes computing an accumulation index value for quantitatively determining the accumulation of the first drug particles in the tissue, based on the spectral information, and determining whether the accumulation index value satisfies the objective condition, and
the determination step includes applying the action to the first drug particles, in a case where the accumulation index value is determined to satisfy the objective condition.

10. The medical device operation method of claim 1, further comprising a confirmation step of executing medical imaging on the treatment target person during and/or after the application of the action, and confirming the treatment effect by the first drug particle, based on medical imaging information acquired by the medical imaging.

11. The medical device operation method of claim 1, wherein the spectral information is
a PCCT image or PCCT scan data collected by PCCT imaging executed by the X-ray CT apparatus,
a PCCT image based on a SECT image or SECT scan data collected by SECT imaging executed by the X-ray CT apparatus, or
a PCCT image based on a DECT image or DECT scan data collected by DECT imaging executed by the X-ray CT apparatus.

12. The medical device operation method of claim 1, wherein the tissue is a cancer tissue.

13. A medical device operation method comprising:
an injection step of injecting a drug particle into a treatment target person;
a monitoring step of executing CT imaging on the treatment target person and monitoring an accumulation of the drug particles in a tissue, based on spectral information acquired by the CT imaging; and
a repetition step of repeating the injection step and the monitoring step until the accumulation of the drug particles in the tissue satisfies an objective condition.

14. The medical device operation method of claim 13, wherein the repetition step includes computing an accumulation index value by image-processing the spectral information, and determining that the objective condition is satisfied, in a case where the accumulation index value reaches a predetermined value.

15. The medical device operation method of claim 13, wherein the repetition step includes determining that the objective condition is satisfied, in a case where a worker inputs an instruction indicating satisfaction of the objective condition via an input device.

16. The medical device operation method of claim 13, wherein the spectral information is
a PCCT image or PCCT scan data collected by PCCT imaging executed by an X-ray CT apparatus,
a PCCT image based on a SECT image or SECT scan data collected by SECT imaging executed by the X-ray CT apparatus, or
a PCCT image based on a DECT image or DECT scan data collected by DECT imaging executed by the X-ray CT apparatus.

17. The medical device operation method of claim 13, wherein the tissue is a cancer tissue.

18. A drug treatment support system comprising:
an X-ray CT apparatus configured to execute first CT imaging on a treatment target person into which a first drug particle with a suppressed onset of a treatment effect is introduced, and to monitor an accumulation of the first drug particles in a tissue, based on spectral information acquired by the first CT imaging; and
an application apparatus configured to apply an action for exerting the treatment effect of the first drug particle to the first drug particle accumulating in the tissue, by using as a trigger an event that the accumulation of the first drug particles in the tissue satisfies an objective condition.

19. A drug treatment support system comprising:
an injection apparatus configured to inject a drug particle into a treatment target person; and
a monitoring apparatus configured to execute CT imaging on the treatment target person and to monitor an accumulation of the drug particles in a tissue, based on spectral information acquired by the CT imaging, wherein
the injection apparatus and the monitoring apparatus repeat the injecting and the monitoring until the accumulation of the drug particles in the tissue satisfies an objective condition.
